(19) United States
(12) Patent Application Publication
(10) Pub. No.: US 2003/0104453 A1
(43) Pub. Date: Jun. 5, 2003

(54) System for pharmacogenetics of adverse drug events

(76) Inventors: David Pickar, Chevy Chase, MD (US); Jack D. Hidary, New York, NY (US); Elizabeth Gray, Cabin John, MD (US)

Correspondence Address: MINTZ LEVIN COHN FERRIS, GLOVSKY AND POPEO PC, 12010 Sunset Hills Road, Suite 900, Reston, VA, 20190-5839, US

(21) Appl. No.: 10/288,338

(22) Filed: Nov. 6, 2002

Related U.S. Application Data
(60) Provisional application No. 60/334,248, filed on Nov. 28, 2001.
(60) Provisional application No. 60/338,541, filed on Nov. 6, 2001.

(51) Int. Cl. C12Q 1/68; G06F 17/60; G06F 19/00; G01N 33/48; G01N 33/50

(52) U.S. Cl. 435/6; 702/20; 705/3

(57) ABSTRACT

The present invention relates to computer systems and methods of analyzing an association between genotypes and adverse drug events for providing personalized medical advice and pharmacogenomic therapy based on patients personal genetic make-up. According to one embodiment, the present invention may create a patient interface for pharmacogenetic studies targeting adverse events and to a database system which allows for application of genetic risk factors for a specific adverse event to a population who might be candidates for specific drug treatment. According to another embodiment, the present invention may provide assistance and guidance in managing and minimizing risk of adverse events utilizing a pharmacogenetic process.

### CROSS REFERENCE TO RELATED APPLICATIONS

This application claims priority from U.S. provisional patent application serial number, 60/334,248, filed on Nov. 28, 2001, and serial No. 60/338,541, filed on Nov. 6, 2001 each of which is incorporated by reference in its entirely.

### FIELD OF THE INVENTION

The present invention relates to computer systems and methods of analyzing an association between patient genotypes and adverse drug phenotypes for providing personalized medical advice based on patients personal genetic make-up.

### BACKGROUND OF THE INVENTION

The success of the worldwide genomics efforts will ultimately be measured by the translation of genomic science into clinical products which affect the practice of medicine and the process by which the biotechnology and the pharmaceutical industries develop successful commercial drugs and other therapeutic products. The utilization of genomic and proteomic data to establish new targets by which to screen new chemical entities as prospective therapeutic agents is rapidly becoming mainstream for drug discovery worldwide. The application of genomics to the clinical development and use of drugs, however, is now in its earliest phase. Bioinformatics platforms provide computational and software tools which enable rapid mining of the enormous genetic sequence, mutation and functional data for a given gene. It is estimated that 2 of 5,000 compounds identified from the drug discovery process eventually reach the clinical market. Once a lead drug candidate is chosen for clinical development, the clinical trial process involves Food and Drug Agency in the United States (FDA) oversight for Phases I-III. Following successful completion of the clinical trial process, the data are submitted to the respective regulatory agency (eg., FDA) as part of the New Drug Application (NDA) process. Regulatory scrutiny, however, does not end with the FDA approval for a drug to be introduced into the market. Post-marketing surveillance (PMS) is, in essence, an ongoing clinical trial in the Phase N category. Although identification and categorization of adverse events is a critical element throughout all Phases of the clinical trial process, the total population exposed to a drug in clinical development typically ranges from 1,000-3,000 people. While extensive, this sample size does not account for the potential side effects that could occur in the tens or hundreds of thousands (or millions) of people taking the drug when it is available for administration to the general population. Moreover, a pharmaceutical company may be required to conduct a Phase IV study, usually in untested populations such as children and the elderly, to extend approved indications into age specific areas.

Pharmacogenomics, the use of genomic information to guide clinical pharmacotherapy and improve outcome has application in all Phases of the Drug Development Life Cycle. Concepts of using pharmacogenomics to guide clinical trials are generally known (see e.g., U.S. Patent Publication 2001/0034023 A1 to Stanton J R, et al., which is incorporated herein by reference in its entirety). The specific application of pharmacogenomics of adverse events (in contrast to genetic identification of high therapeutic responders) includes the post-market surveillance (Phase N) period of the drug life cycle when unexpected adverse events are most likely to arise as well as during early clinical trials. Fundamental to the process of pharmacogenomics has been the establishment of bioinformatics systems designed to maintain, manage and interpret biological data. One drawback in existing systems is a lack of bioinformatics technology to establish a system of databases for individual patients that includes their personal, clinical and genetic data to enable efficient pharmacogenomic therapy. Another drawback in the existing system is a lack of methodologies that provide for establishing individual patient genotypes, including genome wide and candidate gene single nucleotide polymorphisms (SNP's) and detailed adverse drug event information in a unified database to enable the pharmacogenomic therapy.

A key element needed to provide a useful database relating to adverse events is an explicit and consistent definition of adverse event phenotype and polymorphic candidate genes based on understanding the pathways involved in the pathophysiology of the event or based on empirical observation and report without a priori hypothesis. Genetic factors related to individual differences in drug metabolism have long been recognized to affect pharmacokinetics, a key element in tolerability, optimal dose finding and other aspects of pharmacotherapeutics. Thus, genetic factors related to drug metabolism are relevant from early drug development throughout the entire drug life cycle. Therefore, yet another drawback in the existing systems is a lack of bioinformatics system for pharmacogenomic therapy which can utilize genetic factors related to drug metabolic issues.

In addition to metabolic issues, systemic drug adverse events are diverse and have a major impact on the market success of an otherwise successful therapeutic agent. These adverse affects fall under several categories for example: cardiac, liver, central nervous system (including behavior), hematopoetic and metabolic adverse events. A systemic drug adverse event late in the pharmaceutical life cycle (i.e., Phase IV) can be a sudden and limiting factor to a successful product. Therefore, further drawbacks in the existing systems is a lack of bioinformatics system for pharmacogenomic therapy which can utilize systemic drug adverse events.

The pharmacogenomics may also involve the empirical association of numerous relatively low frequency gene variants into a “package” of genetic risk factors which together represent a major tool in the identification of “at risk” populations for a given adverse event. In this way, the small number of patients who might be at risk for even a relatively rare, but medically serious, adverse event might be identified prior to drug administration. This would substantially promote the success of a drug by limiting its adverse affects in its clinical application. However, the existing systems lack bioinformatics features for pharmacogenomic therapy which can analyze low frequency gene variants for adverse drug events.

Other problems also exist.

### SUMMARY OF THE INVENTION

The invention overcomes these and other drawbacks in the existing systems by providing a bioinformatics system for pharmacogenomic therapy that links biological information including genomic and proteomic information for providing personalized medical advice based on a patient's personal genetic make-up.

In one embodiment, the present invention provides an effective system to aid in the identification of patients at risk for systemic drug side effects utilizing pharmacogenetic principles and methods.

In another embodiment, the present invention relates to a relational database which links individualized genomics information to adverse events of therapeutic agents in medicine and provides for its organization and access.

In yet another embodiment, the present invention utilizes bioinformatics technology to establish a system of databases for individual patients, including for example, their personal and genetic data, that enables the identification of genetic risk factors for adverse drug events and its application to clinical trials and market development. In one embodiment, the system provides features for establishing a database of individual patient genotypes, including genome wide and candidate gene single nucleotide polymorphisms (SNP's), and clinical information related to an adverse drug affect experienced by a patient. In another embodiment, the system creates a unified database to enable scientific understanding of risk factors for adverse events and to enable this information to be readily accessible to the clinical trial and clinical market drug development process.

In a further embodiment, the invention provides software to enable a user to select a category of systemic drug side effects, including severity and clinical subtype, the specific mechanism of action of the drug in question within the drug category (e.g., antidepressants, antihypertensives, statins) to receive in an organized format, genetic information such as gene variants and SNP's from public databases, including their allelic frequencies, which have been associated with a given adverse event. In another embodiment, the invention allows for entry of new genetic information or individualized clinical selection criteria that is not necessarily available to the general public.

In an additional embodiment, the invention provides a system for screening patients in clinical trials at all stages (Phase I-N) in order to assess their risk for a specific adverse event for a specific class or individual therapeutic agent. This may enable restricting a pre-approval clinical trial to patients at lowest risk for a known side effect, thereby providing for enhanced “signal to noise ratio.” It may also provide for screening of general populations for adverse event risk factors, thus strengthening the market place of a drug and minimizing the risk for adverse events in the post-market surveillance period (Phase N).

In a further embodiment, the invention provides information regarding a pool of patients (identified anonymously) who have experienced an adverse event to a marketed drug. Such patients may be genotyped for variants of candidate genes relevant to the side effect or class of drug treatment. This may include whole genome-wide SNP data. In this fashion, a unique individualized dataset of clinical populations who have experienced an adverse event can be matched to a corresponding dataset of genetic information.

One aspect of the invention is directed to a system for establishing relationships between genotype (including low frequency SNP's) and adverse events. The system may include, for example, a genotype database, a clinical database, an analytical computer, an adverse event database, a blood bank, sequencing machines and/or clinical indications for applications of specific drugs.

Another aspect of the invention is directed to methods of utilizing genetic variants for high throughput genotyping technologies, including, but not limited to, DNA genotyping and RNA expression “microchip arrays.” The invention is further directed to methods of selecting individual patients who may be at risk for the administration of a specific drug or class or drug by analyzing the genotypes of the patients.

Other objects and features of the present invention will become apparent from the following detailed description considered in connection with the accompanying drawings that disclose embodiments of the present invention. It should be understood, however, that the drawings are designed for purposes of illustration only and not as a definition of the limits of the invention.

### BRIEF DESCRIPTION OF THE FIGURES

FIG. 1 illustrates a pharmacogenomic therapy process for adverse drug events according to one embodiment of the invention.

FIG. 2 illustrates a database system of pharmacogenomic therapy for adverse drug events according to one embodiment of the invention.

FIG. 3 illustrates a system architecture for clinical trial recommendation and pharmacogenomic therapy for adverse drug events according to one embodiment of the invention.

FIG. 4 illustrates the integration of a pharmacogenomics based clinical trial recommendation system, a pharmacogenomic therapy system for adverse drug events and an integrated healthcare management system according to an embodiment of the invention.

FIG. 5 illustrates a process of risk analysis for adverse drug events based on genotypic and drug phenotypic input using pharmacogenomic therapy system according to one embodiment of the invention.

FIG. 6 illustrates an interface for a pharmacogenomic therapy system for adverse drug events according to one embodiment of the invention.

FIG. 7A illustrates an interface for a pharmacogenomic therapy recommendation system according to one embodiment of the invention.

FIG. 7B illustrates an interface for a clinical input of pharmacogenomic therapy recommendation system according to one embodiment of the invention.

FIG. 7C illustrates an interface for a genetic input of pharmacogenomic therapy recommendation system according to one embodiment of the invention.

FIG. 7D illustrates an interface for filtering the inputs of a pharmacogenomic therapy recommendation system according to one embodiment of the invention.

FIG. 7E illustrates an interface for a recommendation information of pharmacogenomic therapy recommendation system according to one embodiment of the invention.

### DETAILED DESCRIPTION OF THE INVENTION

The present invention relates to computer systems and methods of analyzing an association between patient genotypes and adverse drug phenotypes for providing personalized medical advice based on a patients personal genetic make-up. According to one embodiment, the invention may include a user interface for pharmacogenetic studies targeting adverse events to a database system which allows for application of genetic risk factors for a specific adverse event to a population who might be candidates for specific drug treatment. According to another embodiment, the present invention may provide assistance and guidance in managing and minimizing risk of adverse events utilizing a pharmacogenetic process.

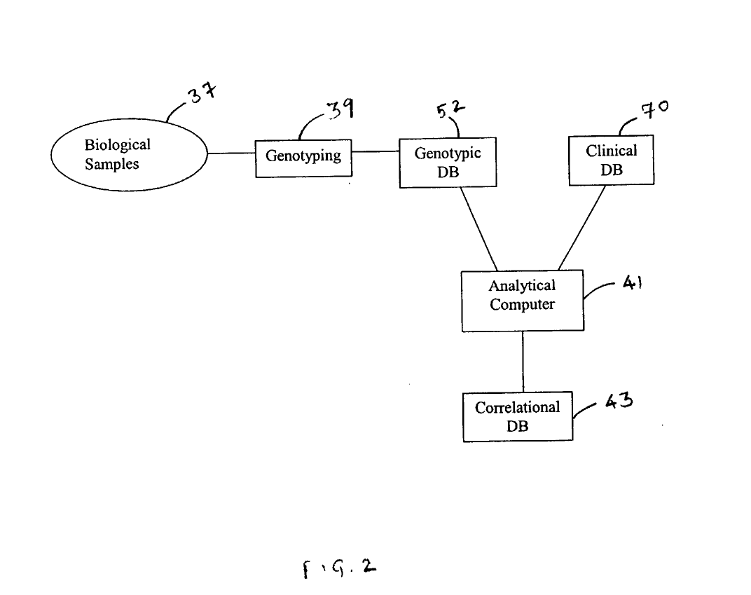

As illustrated in FIG. 2, according to one embodiment of the invention, the database system of the invention may utilize genetic variants to establish a risk for adverse events using principles of pharmacogenetic science.

One or more genotype databases 52 and clinical databases 70 may be merged to establish one or more correlational databases 43, defining genotypic risks for a specific adverse event. One or more genotype databases 52 may be established through collection of biological samples (blood or other tissue) 37 analyzed using a plurality of high throughput genotyping technologies 39. A plurality of associations between contents of genotype databases 52 and clinical databases 70 may be established using an analytic computer 41 producing one or more correlational databases 43.

The one or more genotype databases 52 may include or otherwise access databases that store genotype data. Such data may include, but is not limited to, groups of individual patients who have experienced a specific adverse event to drug treatment and in whom genotype analysis for common and rare variants, including single nucleotide polymorphisms (SNP's) have been determined for specific candidate genes or have been established by a whole genome wide scan. In one embodiment, the sources for the one or more databases may include, for example, proprietary information from a user. In another embodiment, the sources for one or more databases may include, for example, public or open source information (e.g., GenBank). In yet another embodiment, the sources for one or more databases may include, for example, propreitory subscription information (e.g., Incyte Genomics Inc, Celera Genomics Corporation).

One or more clinical databases 70 shown in FIG. 2 may include or otherwise access databases designed to store clinical data. Such data may include, but is not limited to, documented adverse events including the drug which incurred the adverse events, the severity and form of the adverse event (e.g., weight gain, drug-induced prolongation of QTc cardiac interval) and the outcome (cessation of drug treatment, medical care required etc.) FDA documented adverse event profiles may be readily accessible for marketed drugs from many sources including the Physician's Desk Reference from the FDA database.

An analytic computer 41 may refer to a computer that will perform the database analyses described herein. Such a computer may be, for example, a personal computer (e.g., Pentium chip-based), Macintosh computer, Windows-based terminal, Network Computer, wireless device, workstation, mainframe computer, or other computing device. The computer may include, for example, Windows oriented platforms and include conventional software for supporting a display screen, a keyboard, a memory, a processor and input/output device (e.g., mouse). In some embodiments a plurality of analytic computers 41 may be used. In some embodiments, the plurality of computers may be connected as clusters and may be used for parallel processing.

One or more correlational databases 43 may include admixtures of clinical phenotype and genotypic data such that one or more patients may be rapidly selected on the basis of either clinical or genyotypic data to serve the needs of application risk to technologies as part of clinical application (e.g., DNA microarray).

Biological sample collection facility 37 may include a storage means in which whole blood or other tissues are received from patients who enter the database. This facility may allow for the extraction of DNA of leukocytes, immortalization of cell lines for future DNA extraction or the maintenance of tissue for RNA expression studies.

In one embodiment, the genotyping devices 39 may include one or more analytic machines, for example, which provide for high throughput genotyping for individual candidate genes, including “deep sequencing” of large populations for low frequency single nucleotide polymorphisms or other variants. In another embodiment, the genotyping devices 39 may include a plurality of sequencing machines. In some embodiments, high throughput sequencing and genotyping may be acquired through industrial vendors (e.g., Applied Biosystems, Sequenom, Affymetrix) utilizing proprietary technology.

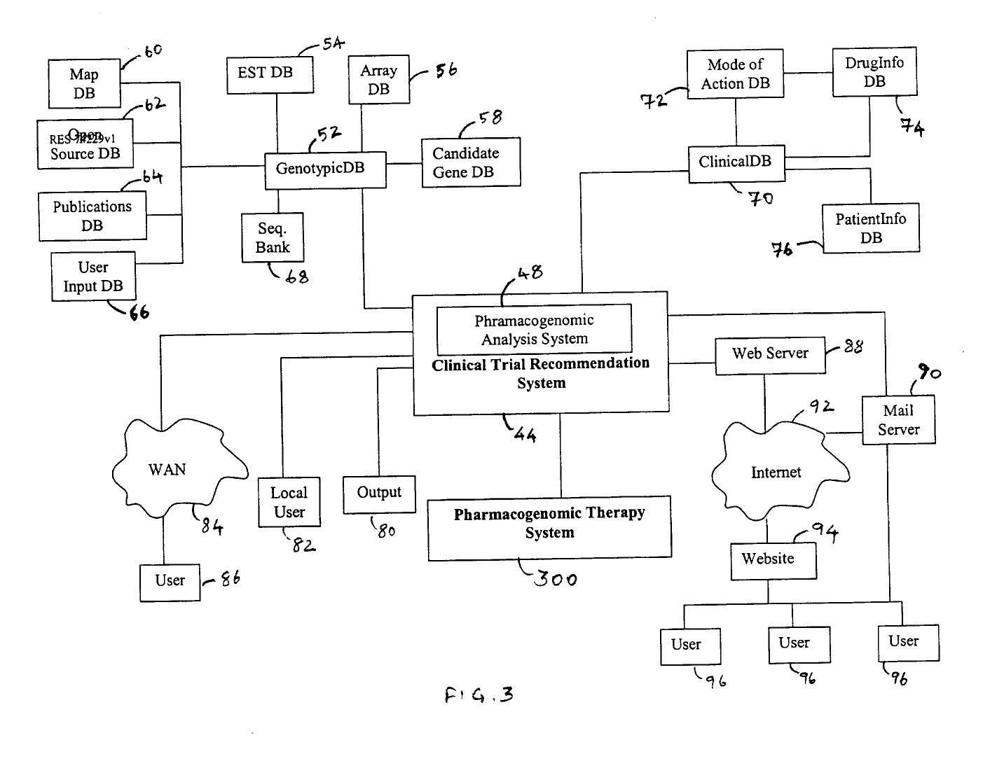

As illustrated in FIG. 3, according to one embodiment of the invention, pharmacogenomic therapeutic system 300 may be coupled to clinical trial recommendation system 44. Clinical trial recommendation system 44 may include pharmacogenomic analysis system 48. Genomic (e.g., associating genotype with phenotype, nucleotide sequence comparison, pattern matching, etc.) and proteomic analysis (e.g., protein sequence matching, three dimensional modeling, etc.) may be performed using pharmacogenomic analysis system 48 of the clinical trial recommendation system 44. The clinical trial recommendation system may include means to access and retrieve genotypic data from, for example, a genotypic database 52 and, clinical data from a clinical database 70.

In one embodiment, the clinical trial recommendation system 44 of the invention may permit the utilization of the genotype data to carry out, design and monitor clinical trials. The one or more genotypic databases 52 may refer to databases designed to store the genotype data. Such data may include, but is not limited to, data associated with groups of individuals or patients in whom genotype analysis for common and rare variants, including single nucleotide polymorphisms, have been determined for distinct candidate genes. This data may also include genome-wide SNP maps for one or more patients. The genotypic database 52 may include or otherwise access expressed sequence information from one or more EST (Expressed Sequence Tag) databases 54, microarray data from one or more array databases 56, candidate gene data from one or more candidate gene databases 58. The one or more genotypic databases 52 may also include or otherwise access genetic sequence (e.g., nucleotide sequence, peptide sequence) from one or more sequence banks 68. The one or more sequence banks 68 may store large volume of genetic data including terra bytes and peta bytes of data. In one embodiment, the one or more sequence banks 68 may access sequence data from a plurality of genetic sequencing devices. In addition, the one or more genotypic databases 52 may be coupled to other databases including, for example, map database 60, open source database 62, publications database 64, and user input database 66. The map database 60 may store information on genetic, physical and transcriptome maps of human and other organisms. The open source databases 62 may include public databases such as, for example, GenBank, SwissProt. The Publications database 64 may include various publications including, for example, subject matters related to genomics, proteomics, and clinical trials. The user Input database 66 may include any information specified by clinical user. The one or more genotypic databases 52 may also be coupled to a plurality of proprietary databases such as, for example, Celera genomic database (not shown in figure).

The one or more clinical databases 70 may include clinical data such as, but not limited to, diagnoses confirmed by standardized assessment tools, confirmed tissue (e.g., tumor) leading to a specific disease diagnosis, illness severity, outcome for illness or syndrome, response to prior drug treatment, family and clinical genetic history, and/or other elements which contribute to a clinical phenotype to be associated with specific genotypes.

The one or more clinical databases 70 may include or otherwise access patient information database 76, mode of action database 72, and/or drug information database 74. The patient information database 76 may include patient information including, for example, medical history, demographical and biographical information (e.g., age, sex). The mode of action database 72 may include, for example, information regarding drug mechanisms. In some embodiments, the mode of action database 72 may include information on partial understanding of a drug mechanism. In other embodiments, the mode of action database 72 may include drug mechanisms which are speculative. The drug information database 74 may include a list of drug manufacturers, dosage information, and results of a previous study.

According one embodiment, the pharmacogenomics based clinical trial recommendation system 44 may include recommended trial database (not shown in Figures). The recommended trial database may include an admixture of clinical phenotype and genotypic data such that a patient, or group of patients, may be rapidly selected on the basis of either clinical or genotypic data to serve the needs of a given clinical trial. In this fashion, a unique database may be applied to a distinct clinical trial.

According to another aspect of the invention, the pharmacogenomics based clinical trial recommendation system 44 may access therapeutic information from one or more pharmacogenomic therapeutic system 300 databases.

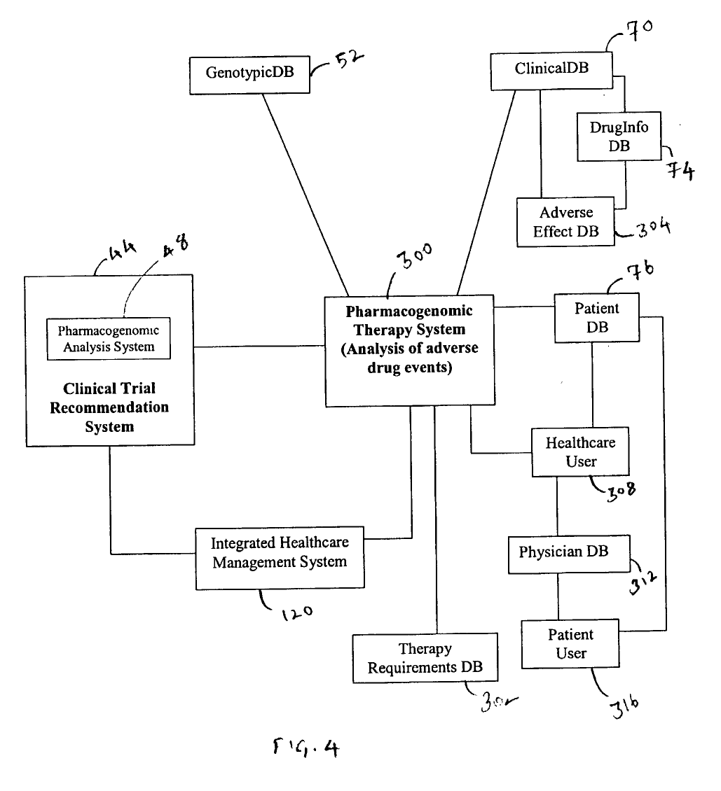

As illustrated in FIG. 4, according to one embodiment of the invention, a pharmacogenomics based clinical trial recommendation system 44, a pharmacogenomic therapeutic system 300 and an integrated healthcare management system 120 may include means to interface and communicate with each other. These systems may have means to access and retrieve genotypic data from one or more genotypic databases 52 and clinical data from one or more clinical databases 70. As illustrated in FIG. 4, the pharmacogenomic therapeutic system 300 may access genetic data from one or more genotypic databases 52, clinical data including adverse drug event data from one or more adverse event databases 304 through one or more clinical databases 70 and patient data from one or more patient databases 76. In some embodiments, the pharmacogenomic therapeutic system 300 may access adverse drug event data directly from one or more adverse event databases 304. Phenotypic characterization of the adverse event may be included in the database to provide insight into the pharmacogenomic processes by which a drug may produce a specific adverse event. Such adverse events may be characterized initially by the affected physiological system (e.g., cardiac, behavioral, endocrine).

According to one embodiment, the one or more clinical databases 70 may access one or more adverse event databases 304 and one or more drug information databases 74. According to another embodiment, the pharmacogenomic therapeutic system 300 may enable patients 316, a plurality of healthcare users 308 such as healthcare managers, paramedical specialists and physicians to access a patient database 76. In some embodiments, this access may be restricted by plurality of authorization means. According to yet another embodiment, the plurality of healthcare users 308 may access pharmacogenomic therapeutic system and analyze genetic data, adverse event data and patient data for providing personalized medicines.

According to another aspect of the invention, the pharmacogenomic therapy system 300 may be integrated with an integrated health care management system 120. The integrated healthcare management system may refer to a system that interacts with one or more organizations for managed care systems (e.g., PPO, HMO), and the plurality of healthcare users 308. The healthcare users 308 may also access clinical trial recommendation system.

In one embodiment, the present invention may permit the utilization of genetic data to gain molecular understanding of adverse events. In another embodiment, the present invention may enable the user to access clinical information about the individual patient's adverse events from a clinical database 70 in relation to that person's individual genomic information. The resultant analyzed database may provide the user with individual patient and/or group information related to an adverse event to a specific drug category (or drug) regarding the genetic associations with the adverse event in relation to genotypes. The system 300 of the invention may provide specific information regarding genotypic relationships between adverse events and specific drug treatments. As such it will be utilized by, for example, pharmaceutical, contract research organizations, site management organizations during clinical development of a new therapeutic agent.

In another embodiment, the invention may allow discovery programs from biopharmaceutical companies to explore genetic relationships to adverse events by providing biological and clinical material from patients in the database who have experienced the adverse event in question. In this way “deep sequencing” efforts (sequencing of large numbers of subjects, e.g., 2 500) may be accomplished in order to identify rare SNP's or other variants related to the adverse event. This information may be utilized by the clinical trial recommendation system 44 to establish a database that could identify new genetic based “targets” for drug discovery programs The risk of adverse events may in this way be minimized early in the small molecule clinical development process.

In yet another embodiment, the present invention may determine an estimate of risk for an adverse event in patients who might be suitable for the therapeutic administration of an approved drug. The user first may enter the drug category (e.g., antidepressant, antihypertensive, antibiotic) and specific therapeutic agent (e.g., fluoxetine, atenelol, Cipro, etc.) for which the patient is a candidate as part of his or her therapeutic regimen. The pharmacogenomic therapeutic system 300 may provide information regarding adverse events and their known association with genetic risk factors for specific drugs or drug categories. The user may also enter the category of adverse event (e.g., cardiac, behavioral) and receive genetic risk factors for the adverse event that may extend across therapeutic agents. The user may then apply a DNA array or other genotyping technologies to biological material from an individual patient in order to gain an estimate of the risk for the adverse event.

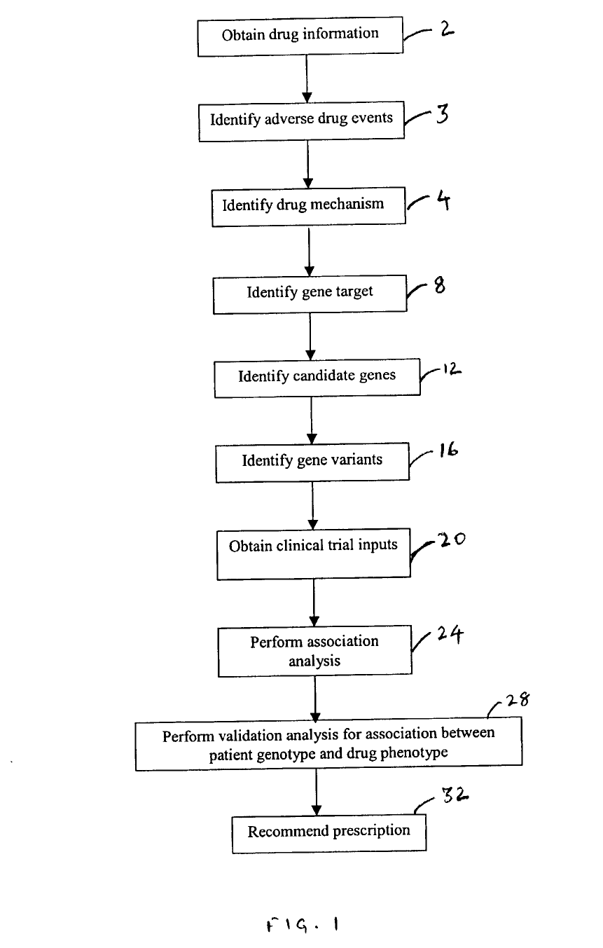

FIG. 1 illustrates, according to one embodiment of the invention, a pharmacogenomic therapy process for adverse drug events using pharmacogenomic information. Components of the pharmacogenomics based therapy may include for example: drug information analysis, adverse drug event analysis, drug mechanism analysis, gene target analysis, candidate gene analysis, gene variant analysis, preliminary clinical trial analysis, association analysis, validation analysis for association, and/or prescription recommendation analysis. Information on one or more of drugs may be obtained as shown in step 2 from one or more drug information databases 74. Similarly one or more adverse drug events may be obtained from one or more adverse event databases 304, as shown in step 3, and adverse events of one or more drugs may be identified and analyzed. Adverse events might include, for example, hypotensive reactions or heart rhythm irregularities (e.g., QTc prolongation), drug-induced diabetes (endocrine) or psychotic reactions (behavioral). In some instances adverse events may involve multiple physiological systems with multiple clinical manifestations.

As shown in step 4 of FIG. 1, drug mechanisms may be identified from one or more mode of action databases 72. The drug mechanisms included in the one or more mode of action databases 72 may provide insight into the pharmacological processes by which a drug produces its therapeutic events. Such drug mechanisms include, for example, information on alterations in function of components of dopamine systems in the central nervous system in the case of antipsychotic drugs, cardiac adrenergic systems for some classes of antihypertensive agents or bacterial genome expression for some antibiotics. In some embodiments, partial understanding of a drug mechanism may be obtained. In other embodiments, information on drug mechanisms which are speculative may be obtained. In yet other embodiments, drug category and information regarding the therapeutic mechanism of action (and known adverse events) of the drug in question may be obtained for the purpose of identifying genetic targets related to the causation of the adverse event. Examples of drug categorization may include, for example, thioridazine, an antipsychotic (a.k.a. neuroleptic) agent within the Phenothiazine chemical group; or the antihypertensive agent, atenolol, a representative of the Benzeneacetamide chemical group, belonging to the therapeutic class of B1-adrenergic blockers; or Cipro (ciprofloxacin), a broad spectrum antibiotic of the fluroquinolone chemical group.

As shown in step 8 of FIG. 1, the present invention enables one to identify gene targets. In one embodiment, gene targets may be included in one or more genotypic databases 52 to provide information regarding a drug's mechanism of action and to provide a basis for pharmacogenomic therapy. In another embodiment, the targets may be included in the one or more genotypic databases 52 to provide information regarding both the drug's mechanism of action and pathophysiological pathway for an adverse event. This might provide the basis for application of pharmacogenetics for risk identification. Such targets may include, for example, striatal D2 receptors for extrapyramidal side effects of antipsychotic drugs or cytochrome P450 for pharmacokinetic variability of the numerous drugs which are metabolized through the cytochrome P450 system.

According to one embodiment, candidate genes of the invention may provide a link between the target (e.g., receptor, enzyme) and genetic control of the target's function and production. These candidate genes may be identified in step 12 from one or more candidate gene databases 58 of the present invention.

According to another embodiment, the invention may include or otherwise access information on gene variants and information on the genetic basis for pharmacogenetics studies. For example, the gene that codes for the D₂ receptor exists with common variants (>1% of the population) in the promoter as well as coding regions. These variants alter an individual's production or composition of the receptor which renders this an excellent target for pharmacogenomic exploration. Common gene variants of specific enzymes of the P450 cytochrome system may enable characterization of patients into three distinct metabolizing patterns: rapid, intermediate and slow. These gene variants may be identified in step 16 from the genotypic database 52 using the pharmacogenomic therapeutic system 300. The gene variants may be due to, but not limited to, SNPs (Single Nucleotide Polymorphisms), variations in candidate genes, variations in number of nucleotide repeats (e.g., simple sequence repeats), variations in length of nucleotide repeats, RFLPs (Restriction Fragment Length Polymorphisms), variations in protein sequences and/or variations in protein structures. In some embodiments gene variants may be scanned over the entire genome. A genome wide scan may enable the search for genetic susceptibility to disease or adverse event without initial focus placed on a specific candidate gene. Scientists using rapidly emerging “haplotype” maps of the genome may more readily be able to “scan” throughout the entire genome for linkages and associations between phenotype and genotype. Haplotypes are ancestral segments of chromosomes that contain many SNP's inherited together as a set or a block enabling easier, faster and less expensive ways to find disease or adverse event causing or predisposing genes which may be characteristic of individual patients. Genome wide scans may be performed on data in the genotypic database 52 for enabling the assembly of a detailed haplotype (SNP block) profile for the adverse event.

According one embodiment, the clinical trial recommendation system 44 may obtain clinical trial information as shown in step 20 and perform association analysis using the genotypic and the phenotypic input. According to one aspect of the invention, an association may be established in step 24 between one or more gene variants and one or more phenotypes (e.g., adverse response to drug, drug mechanisms). Once the association is determined through association analysis in step 24 of the system components, a priori hypothesis testing in further clinical trials can be accomplished. According to one embodiment of the invention, the association may be determined using a plurality of statistical methods. In one example, a pearson's correlation may be used to determine the association between a genotype and clinical phenotype. According to another embodiment of the invention, the associated patient genotype and drug phenotype may be validated in step 28 using one or more statistical methods known to one skilled in the art.

According to another embodiment, clinical and genetic data may be admixed into the one or more correlation databases 43. Once a relationship is established between one or more genotypes and one or more adverse events through association analysis of the database components, the information may be used to develop screening or other clinical monitoring techniques to identify patients who might be at risk for experiencing the adverse event. Numerous SNP's and other candidate gene variants may be assembled onto a DNA microarray “chip” or other technologies which may enable rapid multiple genotyping for one or more individual patients, thereby creating a clinical efficient and validated method for establishing pharmacogenetics risk for an: adverse event. This methodology may then be applied broadly as a clinical screening tool for patient populations.

According to one aspect of the invention, the clinical trial recommendation system 44 may be able to bring genetic information and clinical information of associated genotypes and phenotypes. These associations may be filtered using a pre-determined statistical significance or threshold value. In one embodiment, the information may be filtered based on genes. For example, a user may be interested in a particular gene selected from several genes showing association for a clinical trait. In this case, the user may be able to select one or more preferred genes and filter out the genes and the information related to the genes which are not preferred. In another embodiment, the information may be filtered based on one or more preferred phenotypes. In yet another embodiment, the information may be filtered based on one or more preferred associations between one or more genotypes and one or more phenotypes. According to another aspect of the invention, information on associations and validated associations may be used for further analysis for recommending prescriptions in step 32.

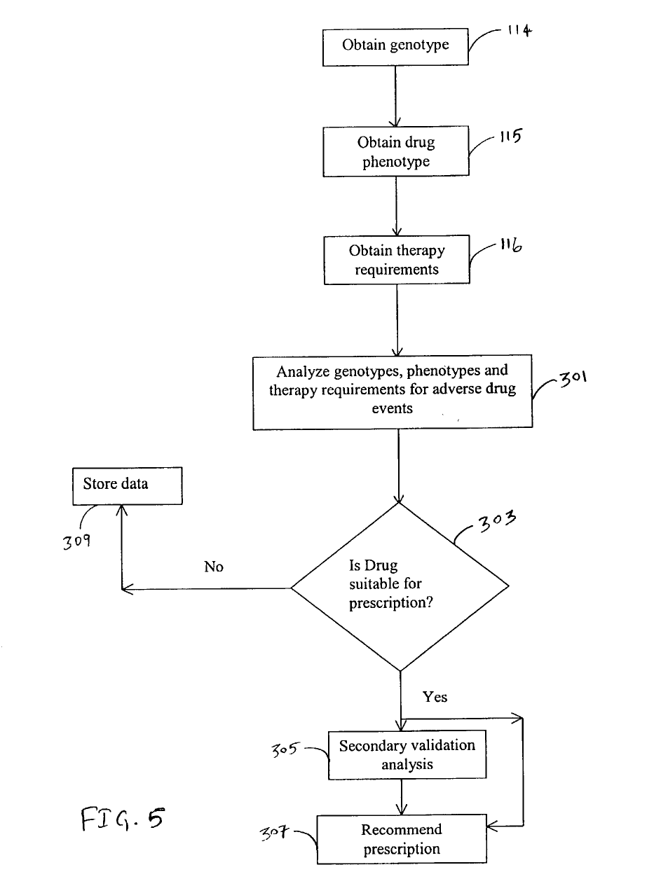

According to one aspect of the invention, the process of analysis for a therapy based on genotypic and drug related phenotypic information and recommending a drug as prescription is illustrated in FIG. 5. In one embodiment, a plurality of genotypes 114, and a plurality of drug related phenotypes 115, may be analyzed using one or more analytical processors. The drug related phenotype may refer to traits such as response to drug, dosage of drug, adverse event of drug, severity of adverse events, etc. In this analysis, individuals having similar genotypes and similar drug related phenotypes may be selected and grouped together. One or more selective genotypes may be associated with one or more selective phenotypes. Means for inclusion and exclusion of selected genotypes and phenotypes may be provided. These inclusions and exclusions may depend on nature of a therapeutic analysis. In one embodiment, genotypes with high similarity may be included for a therapeutic analysis. In another embodiment, genotypes may be randomly chosen to have genetic balance, and included in a therapeutic study. In a further embodiment, the invention provides for ongoing patient selection balance. This involves maintaining balanced treatment “arms,” involving patients with specific genotypes, wherein the system ensures sufficient statistical power needed for hypothesis testing.

In one embodiment, the selected genotypes and drug related phenotypes may be analyzed with the patient related information (e.g., age of patient, health history of patient, etc.). In another embodiment, the selected genotypes and drug related phenotypes may be analyzed with the therapy requirements. Therapy requirements may include, for example, classes of medication, choice of specific medication, etc. In yet another embodiment, the selected genotypes and drug related phenotypes are analyzed with clinical trial data including plurality of clinical trial requirements of individual phase (e.g., Phase III) of a clinical trial.

According to one aspect of the invention, the pharmacogenomnic therapy system 300 may obtain data on therapy requirements from the plurality of therapy requirements database 302, as shown in 116. In some embodiments, the pharmacogenomnic therapy system 300 may include means to select genotypes and analyze drug phenotypes with the plurality of therapy requirements. Associations among the selected genotypes, the drug phenotypes and the therapy requirements may be determined using one or more algorithmic methods (e.g., hidden-markov based analysis, artificial intelligence and neural network, etc.,). The associated genotypes, phenotypes and therapy requirements may be further analyzed for risk for adverse drug events using one or more pre-determined formulas or algorithms, as shown in 301. If there is no risk for adverse events and the selected drugs are suitable for prescription, the analysis may be validated using a plurality of statistical validation models known to one skilled in the art. In some embodiments, the analysis results may be validated against the plurality of clinical trial requirements of individual phase (e.g., Phase III) of a clinical trial. In other embodiments, the invention provides a system for screening patients in clinical trials at all stages (Phase I-N) in order to assess their risk for a specific adverse event for a specific class or individual therapeutic agent. This may enable restricting a pre-approval clinical trial to patients at the lowest risk for a known side effect, thereby providing for enhanced “signal to noise ratio.” It may also provide for screening of general populations for adverse event risk factors, thus strengthening the market place of a drug and minimizing the risk for adverse events in the post-market surveillance period (Phase N).

According to another aspect of the invention, as shown in step 303 of FIG. 5, the system 300 may determine whether or not a selected drug is suitable for prescription for one or more diseases or disorders of a selected individual based on the results of the analysis of adverse drug events (discussed above). If the selected drug is not suitable for prescription, the results of the analysis may be stored, as shown in FIG. 5. If the selected drug is suitable for prescription, the system may perform additional validation or secondary validation of this prescription using one or more user selectable validation models which are not used in previous analysis, as shown in an optional step 305. In some embodiments, the system 300 may enable a user to recommend prescription for the selected individual based on one or more of the analysis procedures (discussed above) for adverse drug events, as shown in step 307.

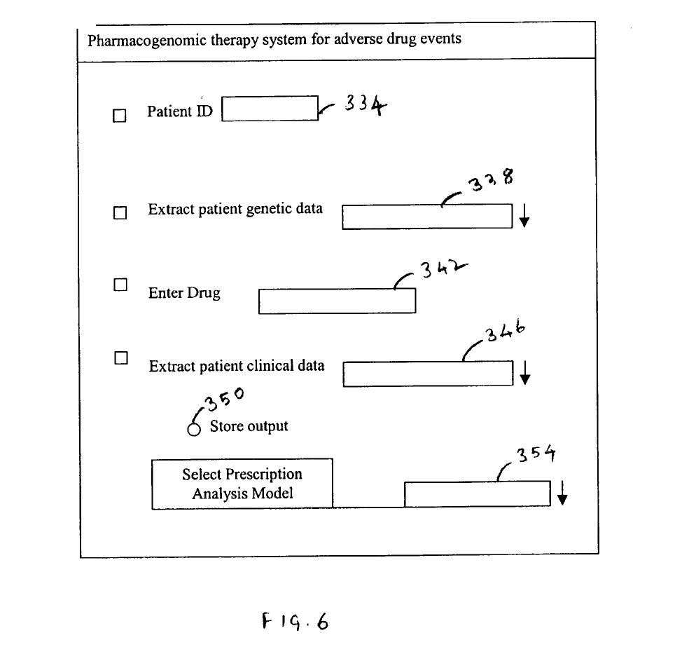

As illustrated in FIG. 6, an interface for the pharmacogenomic therapy recommendation system 300 for adverse drug events may include means for enabling a user to enter, for example, patient information, means for extracting and analyzing patient genetic data and patient clinical data, means for enabling a user to enter drug information and recommend a prescription utilizing a plurality of prescription analysis models. The user may enter a patient ID in interface portion 334 and obtain patient related information. The user may obtain specific information about a patient. Patient genetic data may be obtained by clicking one of the options in scroll down menu box 338. These options may include, but are not limited to, SNP (single Nucleotide Polymorphism) variants, candidate gene variants, simple sequence repeat variants and protein structure variants. Patient clinical data may be obtained using box 346. The examples of patient clinical data may include, for example, patient health history, age, and demographical information. In one embodiment, the user may enter one or more drugs in box 342 and retrieve adverse events of the entered drugs. The user may perform risk analysis of the entered drugs for adverse drug events. The user may store output of the analysis using item 350. Prescription recommendation analysis for pharmacogenomic therapy may be performed by selecting one or more prescription analysis models provided in 354. These models may include statistical or mathematical methods which utilize information from patient genetic data, patient phenotype data and selected drug data for predicting risk for adverse drug events. In one example, artificial intelligence and neural network model is used for prescription recommendation. In another example, principal component analysis is used for prescription recommendation. In yet another example, combinatorial matrix approach is used for prescription recommendation. In one embodiment, the user may have options for selecting one of the pre-determined statistical or mathematical models.

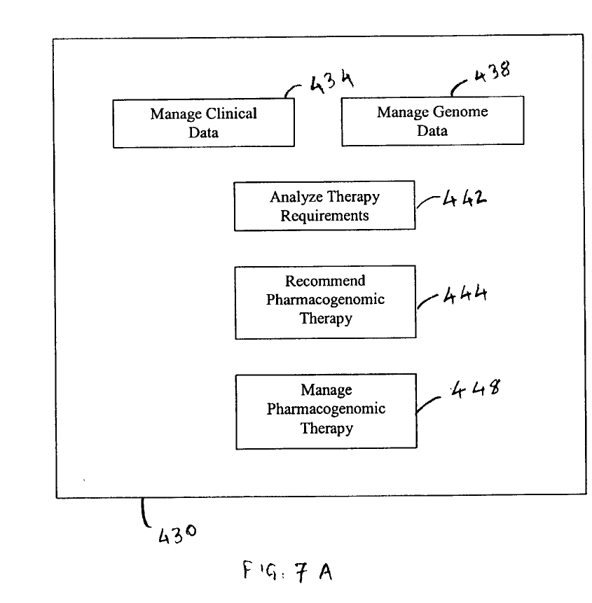

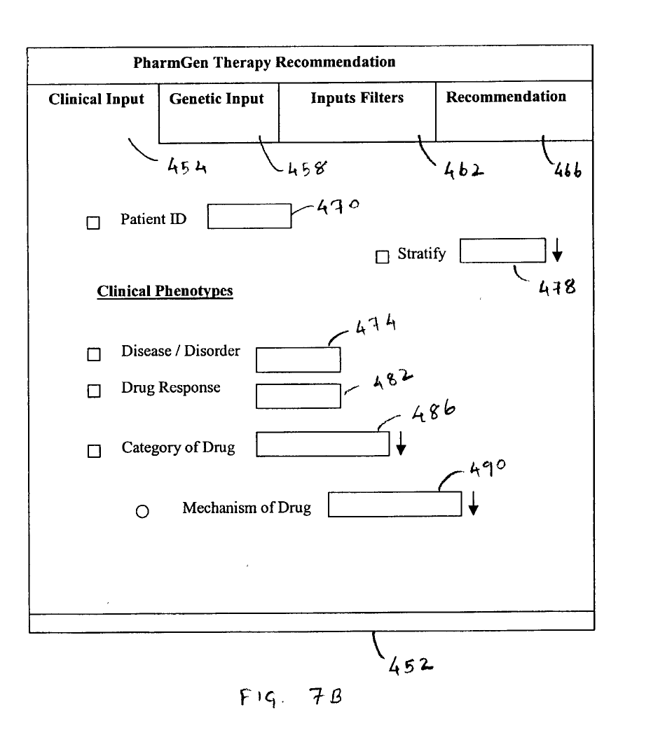

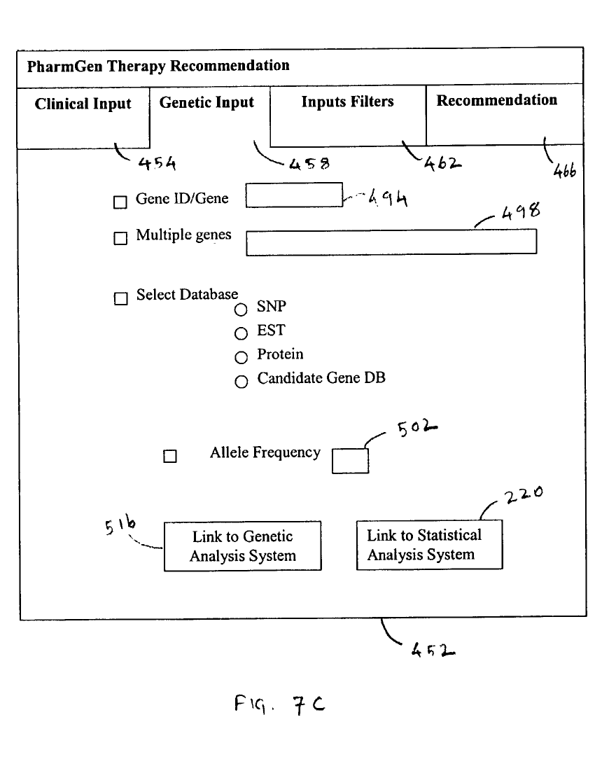

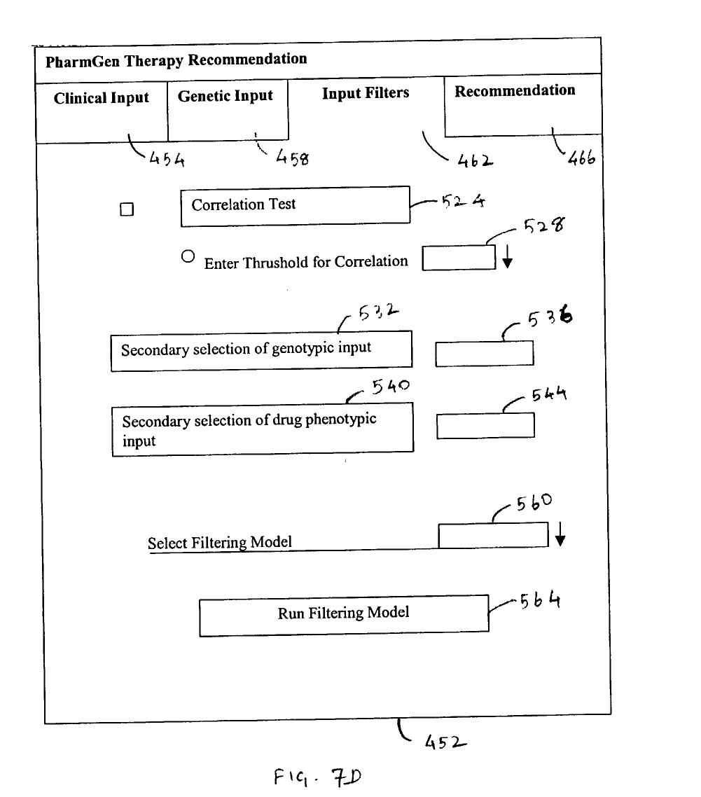

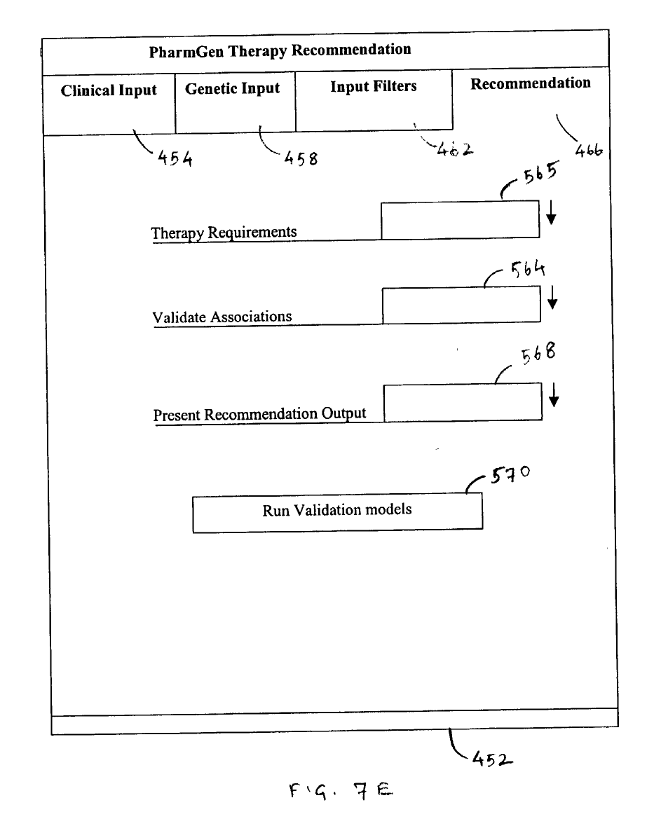

FIG. 7A illustrates a user interface 430 for pharmacogenomic therapy recommendation system 300. The user interface 430 may include a plurality of inputs (i.e. clickable buttons) for managing clinical data 434, managing genomic data 438, analyzing therapy requirements 442, recommending pharmacogenomic therapy 444 and managing pharmacogenomic therapy 448. Manage clinical data button 434 may enable a user to access maintenance features of pharmaceutical, patient, and/or other clinical phenotypic databases in the system 44. Clinical database maintenance features may include entry and editing of data in the clinical databases. The relationships among data and databases may also be managed using these features. In one embodiment, the clinical database management features may include user intervened data update features. In another embodiment, the clinical database may be managed and updated automatically without user intervention. In some embodiments, the clinical database management features may include, for example, plurality of frames preferably in graphical user interface for performing database maintenance functions.

Manage genome data button 438 may enable a user to access genetic data (e.g., nucleotide sequence, protein sequence, protein structural data, protein functional data, genome map) and publications and reports relevant to genetic data of, for example, both proprietary and public databases. Furthermore, the user may operate genome database management features through button 438 for entering and editing of data in the genomic or genetic databases of the system 44. For example, the user may manage the relationships among genetic data and databases. In one embodiment, the genome database management features may include user intervened data update features. In another embodiment, the genome database may be managed and updated automatically without user intervention. In some embodiments, the genome database management features may include a plurality of frames preferably in graphical user interface for performing database maintenance functions.

Pharmacogenomic therapy requirements may be analyzed using button 442. This button 442 may enable a user to access a plurality of frames (not shown in figure), wherein information on therapy requirements of a plurality of diseases/disorders may be recorded. In some embodiments, the system may include a pre-determined format for entering therapy requirement information. In other embodiments, the user may create the formats. These formats may correspond to requirements specified by healthcare organizations.

Manage pharmacogenomic therapy button 448 may be coupled to database management features (not shown in FIG. 7A) to manage data during the therapy. For example, the health status of patient, diagnoses, treatments, and outcomes may be managed. According to one embodiment, pharmacogenomic therapy database management features may support data import from other data systems containing patient data. A plurality of import/edit screens may be used to for pharmacogenomic therapy database management.

When a user clicks pharmacogenomic therapy recommendation 444, the system enables the user to view an interface for pharmacogenomic therapy recommendation 452 as illustrated in FIGS. 7B, 7C, 7D, and 7E. The interface 452 may include features for inputting clinical and genetic information, filtering the information and may provide a recommendation for pharmacogenomic therapy. For example, the interface 452 of FIGS. 7B, 7C, 7D, and 7E may include user selectable frames such as clinical input 454, genetic input 458, input filters 462 and recommendation 466 in the graphical user interface. According to one embodiment, a plurality of clinical phenotypic records may be obtained, analyzed and managed using clinical input frame 454 as illustrated in FIG. 7B. The clinical input interface 454 may provide a plurality of options for the user to select one or more clinical phenotypic traits. The examples of the clinical phenotypic traits may include diseases (e.g., Alzheimer), disorders (e.g., cognitive impairment), drugs (e.g., dopamine), categories of drugs (e.g., antidepressant, anti-hypertensive agents), mechanisms of drugs (e.g., serotonin reuptake inhibitor antidepressant; ACE inhibitor antihypertensive). As illustrated in FIG. 7B, according one embodiment, the user may enter a patient ID in box 470 and retrieve individual patient data including patient phenotypic data from patient database 76 of the system 44. In another embodiment, the user may select a clinical phenotypic trait and analyze clinical phenotypic information of a group of patients using clinical input frame 454. For example, the user may enter disease phenotype in box 474 and retrieve disease data from the clinical database 70 of the system 44. The disease data may include, but is not limited to, symptoms of disease, diagnostic information and treatment information. Similarly, the user may enter a drug response phenotype in box 482 and retrieve drug data from the drug information database 74 of the system 44.

According to another aspect of the invention, the user may input drug related information such as, for example, a category of drug, mechanism of drug, etc. In one embodiment, the user may select a drug category from scroll down menu 486. In another embodiment, the user may select drug mechanism using scroll down menu 490. The system 44 may have means to obtain the information related to selected drug category or drug mechanism from the drug database 74. In addition, the user may stratify the selected clinical phenotypic traits based on a plurality of statistical models known in the art for stratification. The user may use scroll down menu 478 for selecting a statistical model for stratification. In one embodiment, the statistical model for stratification may correspond to phenotypic correlation of individuals. In another embodiment, the statistical model for stratification may correspond to chi-square methodology for grouping individuals. Stratification of individuals based on their clinical phenotypic traits may enable physicians to therapy information for a group of individuals with similar clinical phenotype.

The invention allows for the user to enter information regarding genetic markers that pertain to biological mechanism of a specific drug undergoing clinical trial. The end result of this tool is to balance distributions of genotypes among study populations undergoing specific clinical trials. Thus, the ability to monitor composition of clinical trial populations during the conduct for the clinical trial is provided for.

According to one embodiment, the relational database of the invention may enable the selection of individual patients who are suitable for a clinical trial on the basis of already performed genotypes.

The genetic input of clinical trial recommendation is illustrated in FIG. 7C. According to one aspect of the present invention, the genetic input frame 458 may enable the user to select one or more genetic input means from a plurality of genetic input means. In one embodiment, the user may enter gene identification number or name of gene in box 494 and obtain a plurality of information related to the specified gene from the genotypic database 52 (shown in FIG. 3). In another embodiment, the user may enter more than one gene or multiple genes in box 498 and obtain information related to multiple genes from genotypic database 52 (shown in FIG. 3). The information on multiple genes may correspond to clinical studies of complex diseases since the complex diseases are known to be controlled by multiple genes. In yet another embodiment, the user may select plurality of database sources for obtaining genetic data. The genetic data may include, but is not limited to, SNP (single nucleotide polymorphism), EST (Expressed Sequence Tags), protein data, and candidate genes. These data may be obtained from one or more databases such as, for example, Seq. Bank 68, EST DB 54, and candidate gene DB 58 of system 44. The genetic input frame 458 may include a link to a genetic analysis system 516, wherein the genetic analysis system 516 enables the user to perform genomic (e.g., sequence matching and gene identification, gene expression analysis, genotype analysis) and proteomic (protein identification, predicting protein structure, predicting protein-protein interactions) analysis. The genetic input frame 458 may also include a link to a statistical analysis system 220, wherein, the statistical analysis system 520 enables the user to analyze genetic data using plurality of statistical or mathematical methods (e.g., principal component method for gene expression, regression methods for genotype association, Hidden-Markov methods for sequence matching, etc.). The statistical analysis system may also include means or features for grouping or stratifying individuals based on a plurality of genetic similarities. In some embodiments, the selected genes may be allelic variants. The allele frequency selected genes may be displayed in box 502.

The pharmacogenomics may also involve the empirical association of numerous relatively low frequency gene variants into a “package” of genetic risk factors which together represent a major tool in the identification of “at risk” populations for a given adverse event. In this way, the small number of patients who might be at risk for even a relatively rare, but medically serious, adverse event might be identified prior to drug administration. This would substantially enable the success of a drug by limiting its adverse affects in its clinical application. In one embodiment, the invention enables the user to identify and store one or more genotypes whose allele frequencies are below a pre-determined limit (not shown in figure).

According to another aspect of the invention, as illustrated in FIG. 7D, the user may associate the selected genetic inputs with the selected clinical phenotypic inputs. These associations may be determined using one or more of statistical tests. For example, the user may perform correlation test as shown in box 524 of FIG. 7D. The association may be performed between one or more genes including allelic variants and one or more clinical phenotypic traits. The user may filter the associations using a plurality threshold levels for selecting the associated samples. For example, in one embodiment, the threshold level for correlation may be selected from box 528. In some embodiments, the threshold levels may be pre-determined. Healthcare users including physicians and researchers may be interested in focusing on few genes or selecting few genes. Similarly, they may be interested in few aspects of information relevant to phenotypic traits. According to one embodiment of the invention as illustrated in FIG. 7D, the user may filter the selected clinical and genetic inputs and the retrieved information related to the selected clinical and genetic inputs. The genetic input may be further selected from box 532. The further selected genetic input may be displayed in box 536. Similarly, the clinical phenotypic input may be further selected from box 540 and the further selected phenotypic input may be displayed in box 544. According to one embodiment of the invention, the user may filter the inputs using one or more filtering models in 560. The filtering models may be run using box 564. The filtering models may include parameters such as threshold level for association between genetic input and clinical input, threshold level for similarity between the selected genetic or phenotypic input and the retrieved information from one or more databases in the system 44. According to one aspect of the invention, when the user knows which candidates are pertinent to the drug trial, the clinical trial recommendation system 44 may enable the choice of specific patients, already categorized by patterns of candidate gene variants and/or single nucleotide polymorphism (SNP) patterns, thereby enabling the organizers and managers of clinical trials to establish and select pre-hoc trial populations which enable hypotheses of genetic variants as predictors of therapeutic response to be tested in an efficient and scientifically rigorous fashion.

According to another aspect of the invention, the system provides optimization features for clinical trials. As illustrated in FIG. 7E, the user may select one or more therapy requirements using box 565. These therapy requirements may obtain data from therapy requirements database 302. The associated genotype and drug phenotypes may be analyzed using therapy requirements to enable pharmacogenomic therapy for plurality of patients.

In one embodiment, the associations for pharmacogenomic therapy may be validated using plurality of validation models in box 564. These validation models may be statistical or mathematical models including but not limited to artificial intelligence and neural network methods, maximum likelihood methods, principal component methods, and combinatorial matrix algorithms. These models may include genotypic variables, phenotypic variables and variables related to one or more therapy requirements specified by the user. In some embodiments, these methods may correspond to features or means that could convert qualitative information into quantitative variables and may include such variables in validation.

According to another embodiment, the user may obtain plurality outputs for therapy recommendations. In one embodiment, the user may be able to select one or more presentation formats from box 568. After selecting the required elements for validation of recommendation, validation models may be run by clicking box 570.

The present invention may enable healthcare users including physicians, researchers and clinicians to gain information regarding genetic risk factors for specific adverse events related to individual drugs across therapeutic categories and provides a mechanism of applying this information to the patient. It may also provide for the use by companies wishing to establish proprietary diagnostic tools based on findings from internal discovery programs or specialized information related to their drugs.

While a particular embodiment of the present invention has been described, it is to be understood that modifications will be apparent to those skilled in the art without departing from the spirit of the invention. The scope of the invention, therefore, is to be determined solely by the following claims.

This invention will be better understood by reference to the following non-limiting examples.

### EXAMPLE #1

A pharmaceutical company may wish to bring a lead compound targeted as an antipsychotic into clinical trials. The system of the invention can be used to assist in such efforts.

In this example, the compound has already passed through Phase I trials and showed no limiting adverse events in normal controls. Moreover, Phase II trial data suggest that the drug has an impressive antipsychotic profile. It is noted, however, that a potential market limiting adverse event was observed in some, but not all subjects: weight gain >4 kg over 6 weeks of drug administration. It is known that weight gain limits market acceptance and further may predispose diabetes. For this reason, the company wishes to carry out its Phase III trial in a fashion that minimizes weight gain without interference with the signal of the drug's effectiveness. Once efficacy is established, the company wishes to demonstrate that those subjects at risk for weight gain may benefit from co-administration of a second, commercially available agent which otherwise may be of no benefit to the indication (e.g., HI antagonist).

The pharmaceutical company may utilize the pharmacogenomic therapy recommendation system 300 of the invention in the following fashion:

1. The pharmacogenomic therapy recommendation system 300 first provides the company with information regarding drug-induced weight gain including known genetic variants which are thought to represent risk factors.

2. The pharmacogenomic therapy recommendation system 300 also provides information regarding specific genetic associations between weight gain in relation to the category and chemical class of the company's drug undergoing clinical trial.

3. It is learned using the pharmacogenomic therapy recommendation system 300 that variants of the 5 HTzc receptors are associated with weight gain in patients. This becomes relevant because the drug in clinical trial has, as part of its mechanism of action, antagonist properties to the 5 HTzc receptor.

4. It is also learned using the pharmacogenomic therapy recommendation system 300 that there are 200 low frequency SNP's which have been found in individual patients to be associated with excessive weight gain to a number of different drugs.

5. The company desires to target the major emphasis of its Phase III clinical population to exclude patients with risk of excessive weight gain, so as establish clear effectiveness as an antipsychotic without limiting market possibilities.

6. As a secondary goal, the company whishes to conduct a preliminary, controlled trial in patients at risk for excessive weight gain which includes co-administration of an HI antagonist to minimize the anticipated adverse event.

7. The strategy proves to be successful in that the clinical trial excluding patients at risk for excessive weight gain event achieves the desired goal: antipsychotic efficacy in demonstrated with modest group weight gain data (4.5 kg per patient).

8. The company continues with the clinical trial designed specifically for at risk patients and finds that co-administration of restricts weight gain without interfering with antipsychotic efficacy.

9. The NDA application includes genotypic patient information which provides a risk assessment for excessive weight gains dependent upon genotype.

10. The company utilizes the pharmacogenomic therapy recommendation system 300 to develop diagnostic DNA “chip” enabling all patients to be candidates for treatment with their drug and allows for identification of those at risk for weight gain, enabling a strategy of co-administration of a secondary agent.

### EXAMPLE #2

In this prophetic example, a company has successfully marketed a broad spectrum antibiotic. It has become apparent that an unexpected and rare adverse event has emerged which has potentially serious implications for continued market success. This event is the emergence of psychosis and/or other serious behavioral disturbances during treatment >14 days. The drug has now received increasing attention and unanticipated application as a prophylactic agent for the fatal disorder, Anthrax. The drug is administered to many more patients and for a longer period of time than expected. The behavioral adverse event takes on greater significance as this the drug is now widely used for extensive treatment periods in individuals exposed to this toxic agent and enhances fear related to potential anthrax exposure. Because other antibiotics without this adverse event may also be effective in the treatment of Anthrax, the company wishes to identify patients at risk for behavioral adverse events.

The pharmaceutical company may use the pharmacogenomic therapy recommendation system 300 and clinical trial recommendation system 44 to accomplish their goal of establishing genetic risk factors for the relatively rare but limiting adverse event of toxic behavioral disturbance.

1. The company first accesses to the pharmacogenomic therapy recommendation system 300 to learn about known genetic factors which might predispose to psychotic reactions.

2. Some of the information received relates to targets, genes and gene variants known to be associated with the neurobiology of psychosis in general.

3. The clinical trial recommendation system 44 confirms that drug-induced behavioral disturbances occur with treatment with other drug categories, although the known mechanisms (e.g., inhibition of dopamine beta-hydroxylase) do not appear to be related to pharmacological effects of the company's antibiotic.

4. The company decides to utilize the clinical trial recommendation system 44 in two ways. First, it will genotype patients who have experienced psychosis during treatment with the antibiotic for targets though to represent neuronal pathways for psychosis. These include, but are not limited to known variants of genes coding for dopamine receptors (2,3,4) and the dopamine transporter gene.

5. The company also decides to access from the clinical trial recommendation system 44 (and add new patients to the database) patients who have experienced drug-induced psychosis to the antibiotic. The company then proceeds with an intensive deep sequencing program designed to identify heretofore unknown low frequency SNP variants that are associated with drug-induced psychosis.

6. Utilizing the clinical trial recommendation system 44, the company establishes that all individuals who have experienced drug-induced psychosis to the antibiotic show a far higher frequency of functional gene variants of the dopamine transporter than are found in the general population. This leads to the hypothesis that gene variants of the dopamine transporter are a major determinant in providing risk for antibiotic-induced psychosis.

7. The company begins Phase IV trials to prospectively test the hypothesis of the role of the dopamine transporter variant in the induced psychosis produced by its drug.

8. The company further discovers through its sequencing program which utilizes the clinical trial recommendation system 44 database that there is a pattern of low frequency SNP's which also are related to behavioral disturbances produced by the antibiotic.

9. The company initiates the development of a proprietary DNA chip intended to identify patients at risk for behavioral disturbances associated with treatment with its proprietary antibiotic.

### EXAMPLE #3

In this prophetic example, a company has developed a drug which has received approval by the FDA as branded prescription product. During the conduct of Phase III trials, it was noted that mean increases in the QTc EKG interval occur but it was agreed by the FDA, including its advisory panel, to be a drug-induced physiological effect that represented an acceptable risk, comparable to other drugs of varying therapeutic categories which already had received approval. The company's drug proved to be very successful in the clinical market. However, unexpectedly several serious cardiac arrythmias were reported in Phase IV monitoring by FDA; two cases resulted in sudden death. Because of the severity of these adverse events, immediate scrutiny into the drug's safety was launched by both regulatory and company personnel.

The company utilized the clinical trial recommendation system 44 in order to establish a better understanding of genetic risk factors associated with drug-induced arrhythmias in order to “rescue” an otherwise therapeutically sound and commercially successful product.

1. The company's utilization of the clinical trial recommendation system 44 rapidly identified risk factors discovered through studies of Sudden Infant Death Syndrome which may represent occult risk to drug-induced cardiac arrhythmias and other cardiac conduction defects in adults.

2. The clinical trial recommendation system 44 identified variants of the SCN5A sodium channel gene to be associated with cardiac arrhythmias in 30% of its database of druginduced cardiac arrhythmias in adults. Moreover, the clinical trial recommendation system 44 provided a sample of 500 patients who had documented drug-induced prolongation of the QTc interval and thus were at high risk for cardiac arrhythmia and sudden death.

3. The drug-induced QTc population patient group was suited for prospective investigation into genetic cardiac risk factors which could have implications to the company's drug.

4. The company rapidly performed genotyping for the SCN5A gene on subjects who had experienced drug-induced arrhythmia in Phase IV monitoring. The company found a frequency of 40% in this group, far above the general population frequency of <2%. This information provided a first step to developing a strategy to “rescue” their drug.

5. The clinical trial recommendation system 44's biological database was utilized by the company to extend its research. In addition to the SCN5A, the company discovered through deep sequencing programs that SNP's in other ion channel related genes were found in the drug-induced QTc prolongation population.

6. The company returned to the original group of patients who had experienced cardiac arrhythmias to its drug and examined the frequencies of the newly discovered SNP's. Several but not all of the SNP's were found in the samples.

7. The company initiates a Phase IV trial utilizing these pharmacogenetics findings to validate its hypothesis that these genes represent risk factors for the adverse event of their drug.

8. The FDA's post marketing surveillance policy supports the company's strategy regarding identification of genetic risk factors.

9. The company requests the services of a DNA array chip manufacturer to design a “chip” expressively for the purpose of identifying patients at risk for drug-induced cardiac arrhythmia produced by their drug.

10. The company enters into discussion with the FDA regarding the approval process for this DNA chip as a diagnostic entity.

We claim:

1. A pharmacogenomic system for predicting a risk of adverse events to one or more drugs for a plurality of patients, the system comprising:
a genotype database (GDB), the GDB comprising genetic information for a plurality of patients;
a adverse drug event database (AEDB), the AEDB comprising adverse drug event phenotypic information of for a plurality of patients;
an association module connected to GDB and AEDB and adapted to enable a user to determine an association between the genetic information and the adverse drug event phenotypic information for a plurality of patients;
a risk prediction module that enables a user to predict a risk for adverse drug events for a plurality of patients, wherein the risk prediction module utilizes the determined association between the genetic information and the adverse drug event phenotypic information;
a validation module that enables a user to validate the predicted risk for adverse drug events for a plurality of patients; and
a recommendation module that enables a user to recommend prescription utilizing the validated information for risk for adverse drug events for a plurality of patients.

2. The system of claim 1 further comprising a selection module for selecting one or more patients based on the genetic information, wherein the selection is performed using plurality of statistical methods.

3. The system of claim 1, wherein the genetic information correspond to one or more variation in candidate genes.

4. The system of claim 1, wherein the genetic information correspond to plurality of Single Nucleotide Polymorphisms.

5. The system of claim 1, wherein adverse drug events correspond to multiple physiological systems with multiple clinical manifestations.

6. The system of claim 1, wherein the association is determined my one or more of predetermined statistical methods.

7. The system of claim 1, wherein the validation is performed utilizing one or more predetermined mathematical models.

8. A pharmacogenomic system for predicting a risk of adverse events to one or more drugs for a plurality of patients, the system comprising:
a genotype database (GDB), the GDB comprising genetic information for a plurality of patients;
a adverse drug event database (AEDB), the AEDB comprising adverse drug event phenotypic information of for a plurality of patients;
association means connected to GDB and AEDB and adopted to enable a user to determine an association between the genetic information and the adverse drug event phenotypic information for a plurality of patients;
risk prediction means that enable a user to predict a risk for adverse drug events for a plurality of patients, wherein the risk prediction means utilizes the determined association between the genetic information and the adverse drug event phenotypic information;
validation means that enable a user to validate the predicted risk for adverse drug events for a plurality of patients; and
recommendation means that enable a user to recommend prescription utilizing the validated information for risk for adverse drug events for a plurality of patients.

9. The system of claim 1 further comprising selection means for selecting one or more patients based on the genetic information, wherein the selection is performed using plurality of statistical methods.

10. The system of claim 1, wherein the genetic information correspond to one or more variation in candidate genes.

11. The system of claim 1, wherein the genetic information correspond to plurality of Single Nucleotide Polymorphisms.

12. The system of claim 1, wherein adverse drug events correspond to multiple physiological systems with multiple clinical manifestations.

13. The system of claim 1, wherein the association is determined my one or more of predetermined statistical methods.

14. The system of claim 1, wherein the validation is performed utilizing one or more predetermined mathematical models.

15. A pharmacogenomic method for predicting a risk for adverse events of one or more drugs for a plurality of patients, the method comprising the steps of:
enabling a user to access a genotype database (GDB), the GDB comprising genetic information for a plurality of patients;
enabling a user to access a adverse drug event database (AEDB), the AEDB comprising adverse drug event phenotypic information of for a plurality of patients;
enabling a user to determine an association between the genetic information and the adverse drug event phenotypic information for a plurality of patients;
enabling a user to predict a risk for adverse drug events for a plurality of patients, wherein the risk prediction modules utilize the determined association between the genetic information and the adverse drug event phenotypic information;
enabling a user to validate the predicted risk for adverse drug events for a plurality of patients; and
enabling a user to recommend prescription utilizing the validated information for risk for adverse drug events for a plurality of patients.

16. The method of claim 1 further comprising the step of selecting one or more patients based on the genetic information, wherein the selection is performed using plurality of statistical methods.

17. The method of claim 1, wherein the genetic information correspond to one or more variation in candidate genes.

18. The method of claim 1, wherein the genetic information correspond to plurality of Single Nucleotide Polymorphisms.

19. The method of claim 1, wherein adverse drug events correspond to multiple physiological systems with multiple clinical manifestations.

20. The method of claim 1, wherein the association is determined my one or more of predetermined statistical methods.

21. The method of claim 1, wherein the validation is performed utilizing one or more predetermined mathematical models.

22. A processor readable pharmacogenomic medium for predicting a risk for adverse events of one or more drugs for a plurality of patients, said processor readable medium comprising:
a first processor readable program code for enabling a user to access a genotype database (GDB), the GDB comprising genetic information for a plurality of patients;
a second processor readable program code for enabling a user to access a adverse drug event database (AEDB), the AEDB comprising adverse drug event phenotypic information of for a plurality of patients;
a third processor readable program code for enabling a user to determine an association between the genetic information and the adverse drug event phenotypic information for a plurality of patients;
a fourth processor readable program code for enabling a user to predict a risk for adverse drug events for a plurality of patients, wherein the risk prediction modules utilize the determined association between the genetic information and the adverse drug event phenotypic information;
a fifth processor readable program code for enabling a user to validate the predicted risk for adverse drug events for a plurality of patients; and
a sixth processor readable program code for enabling a user to recommend prescription utilizing the validated information for risk for adverse drug events for a plurality of patients.

23. A pharmacogenomic system for predicting a risk of adverse events to one or more drugs for a plurality of patients, the system comprising:
means for providing genetic information for a plurality of patients;
means for providing adverse drug event phenotypic information of for a plurality of patients;
means for enabling a user to determine an association between the genetic information and the adverse drug event phenotypic information for a plurality of patients;
risk prediction means that enable a user to predict a risk for adverse drug events for a plurality of patients, wherein the risk prediction modules utilize the determined association between the genetic information and the adverse drug event phenotypic information;
validation means that enable a user to validate the predicted risk for adverse drug events for a plurality of patients; and
recommendation means that enable a user to recommend prescription utilizing the validated information for risk for adverse drug events for a plurality of patients.
